SEQUENCE LISTING (4 sequences)

SEQ ID NO: 1 - protein, 108 aa, synthetic construct
EIVLTQSPGTLSLSPGERATLSCRASQSVRGRYLAWYQQKPKQAPRLLIYGASSRATGIPDRFSGSGSGTDFTLTISRLEPEDFAVFYCQQYGSSPRTFGQGTKVEIK

SEQ ID NO: 2 - protein, 215 aa, synthetic construct
EIVLTQSPGTLSLSPGERATLSCRASQSVRGRYLAWYQQKPKQAPRLLIYGASSRATGIPDRFSGSGSGTDFTLTISRLEPEDFAVFYCQQYGSSPRTFGQGTKVEIKRTVAAPSVTIFPPSDEQLKSGTASVVCLLNNFYPREAKVQWKVDNALQSGNSQESVTEQDSKDSTYSLSSTLTLSKADYEKHKVYASEVTHQGLSSPVTKSFNRGEC

SEQ ID NO: 3 - protein, 122 aa, synthetic construct
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKSPGTTVIMSWFDPWGQGTLVTVSS

SEQ ID NO: 4 - protein, 448 aa, synthetic construct
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKSPGTTVIMSWFDPWGQGTLVTVSSASTKGPSVFPLAPCSRSTSESTAALGCLVKDYFPEPVTVSMNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

(19) United States
(12) Patent Application Publication
(10) Pub. No.: US 2026/0242496 A1
(43) Pub. Date: Aug. 20, 2026

(54) DENOSUMAB FORMULATION

(71) Applicant: ALVOTECH HF, Reykjavik (IS)

(72) Inventors: Hordur Kari HARDARSON, Reykjavik (IS); Jon Otti SIGURDSSON, Reykjavik (IS)

(73) Assignee: ALVOTECH HF, Reykjavik (IS)

(21) Appl. No.: 19/161,399

(22) PCT Filed: Mar. 2, 2024
(86) PCT No.: PCT/EP2024/055511

§ 371 Date: Sep. 2, 2025

Related U.S. Application Data
(60) Provisional application No. 63/449,624, filed on Mar. 3, 2023.

(51) Int. Cl. C07K 16/28; A61K 9/08; A61K 47/18; A61K 47/26; A61K 47/34

(52) U.S. Cl. CPC C07K 16/2875; A61K 9/08; A61K 47/183; A61K 47/26; A61K 47/34; C07K 2317/21

(57) ABSTRACT

An aqueous pharmaceutical formulation having improved stability includes denosumab and a poloxamer, and preferably a histidine buffer and/or sugar or sugar alcohol. The formulation is for use in treating or preventing osteoporosis, loss of bone mass, skeletal-related events associated with multiple myeloma, solid tumor bone metastases, giant cell tumors of the bone or hypercalcemia.

### BACKGROUND OF THE DISCLOSURE

### Technical Field

The present disclosure relates to aqueous pharmaceutical formulations of denosumab.

### Description of the Related Art

Any discussion of related art throughout the specification should in no way be considered as an admission that such art is widely known or forms part of common general knowledge in the field.

Denosumab is a fully human monoclonal IgG antibody that binds with high affinity and specificity to the receptor activator of NF-κB ligand (RANKL). There are two commercial denosumab products, Prolia® and Xgeva® (Amgen Inc.), which contain 60 mg/ml and 70 mg/mL of denosumab, respectively. Prolia® and Xgeva® are both parenteral formulations suitable for administration by subcutaneous injection. The lower concentration denosumab product, Prolia®, is indicated for treatment of osteoporosis on a dosing schedule of one dose every six months, with each 1 mL dose containing 60 mg denosumab, 47 mg sorbitol, 1 mg acetate, 0.1 mg polysorbate 20, and sodium hydroxide for adjusting to pH 5.2, in water for injection. Xgeva® is indicated for the treatment of skeletal events associated with cancer, or cancerous tumors, on a dosing schedule of one dose every four weeks, with each 1.7 mL dose containing 120 mg denosumab, 78.1 mg sorbitol, 1.8 mg acetate, 0.17 mg polysorbate 20, and sodium hydroxide for adjusting to pH 5.2, in water for injection.

Product stability is an essential quality attribute for pharmaceutical formulations that is important for both efficacy and safety and is a regulatory requirement for approval by health authorities. It refers to the retention of chemical, physical, and biological properties and characteristics after manufacture, formulation, transportation, storage, and administration. Stability can be particularly challenging for biologics which are prone to aggregation, degradation, oxidation, isomerization, adsorption, and denaturation, which can be accelerated by environmental stresses such as agitation and variations in pH and temperature.

Surfactants perform a vital role in stabilizing biologics. This role can include protection from interfacial stresses such as interface-induced aggregation, precipitation, and adsorption from exposure to a liquid-air interface and contact with purification and other processing, storage, and delivery equipment.

It is an object of the present invention to provide stable formulations of denosumab.

### BRIEF SUMMARY OF THE DISCLOSURE

Polyoxyethylene (PEO) surfactants, including polysorbates, have been identified as promising surfactants to use in biological formulations. However, the present inventor has found that, surprisingly, polysorbates can be unstable and were seemingly the source of particulates and cloudiness during identification and evaluation of denosumab formulations, especially when co-formulated with a histidine buffer. Applicants have discovered that stable denosumab formulations can be made with a poloxamer surfactant, especially when used in combination with a histidine buffer.

According to a first aspect, the present disclosure relates to an aqueous pharmaceutical formulation, including 50 to 80 mg/mL denosumab, 200 to 300 mM sugar, 1 to 10 mM histidine buffer, 0.005 to 0.1% (w/v) poloxamer, and water, the formulation having a pH of from 5.5 to 6.5. In embodiments, the aqueous pharmaceutical formulation includes 55 to 65 or 65 to 75 mg/mL denosumab, 230 to 250 mM sugar, 4 to 6 mM histidine buffer, 0.01 to 0.05% (w/v) poloxamer, and water, the formulation having a pH of from 6.0 to 6.4. In further embodiments, the aqueous pharmaceutical formulation includes 60 or 70 mg/mL denosumab, 240 mM sugar, 5 mM histidine buffer, 0.03% (w/v) poloxamer, and water, the formulation having a pH of 6.2.

According to a second aspect, the present disclosure relates to an aqueous pharmaceutical formulation including denosumab and a poloxamer. The formulation may optionally include a histidine buffer.

According to a third aspect, the present disclosure relates to a denosumab formulation for parenteral injection, including a sugar or sugar alcohol, a buffer, and a surfactant, wherein the surfactant is a poloxamer. In embodiments, the buffer is a histidine buffer.

According to a fourth aspect, the present disclosure relates to an aqueous pharmaceutical formulation, including denosumab and a surfactant. The formulation exhibits a loss of surfactant concentration of 40% or less, when stored for one month at 25° C. and/or a loss of surfactant concentration of 50% or less, when stored for one month at 40° C.

According to a fifth aspect, the present disclosure relates to a formulation as described herein for use in treating or preventing osteoporosis or loss of bone mass.

According to a sixth aspect, the present disclosure relates to a formulation as described herein for use in treating or preventing skeletal-related events associated with multiple myeloma, solid tumor bone metastases, or giant cell tumors of the bone.

According to a seventh aspect, the present disclosure relates to a formulation as described herein for use in treating or preventing hypercalcemia.

According to an eighth aspect, the present disclosure relates to a method for treating or preventing osteoporosis or loss of bone mass, comprising administering a formulation as described herein to a subject in need of such treatment.

According to a ninth aspect, the present disclosure relates to a method for treating or preventing a skeletal-related event associated with multiple myeloma, solid tumor bone metastases, or giant cell tumors of the bone, comprising administering a formulation as described herein to a subject in need of such treatment.

According to a ninth aspect, the present disclosure relates to a method for treating or preventing hypercalcemia, comprising administering a formulation as described herein to a subject in need of such treatment.

According to a tenth aspect, the present disclosure relates to use of a formulation as described herein in the manufacture of a medicament for treating or preventing osteoporosis or loss of bone mass.

According to an eleventh aspect, the present disclosure relates to use of a formulation described herein in the manufacture of a medicament for treating or preventing skeletal-related events associated with multiple myeloma, solid tumor bone metastases, or giant cell tumors of the bone.

According to a twelfth aspect, the present disclosure relates to use of a formulation as described herein in the manufacture of a medicament for treating or preventing hypercalcemia.

The foregoing paragraphs have been provided by way of general introduction and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

FIG. 1 is a graph showing surfactant concentration over time for several inventive and comparative denosumab formulations stored at 5° C.;

FIG. 2 is a graph showing surfactant concentration over time for several inventive and comparative denosumab formulations stored at 25° C.; and

FIG. 3 is a graph showing surfactant concentration over time for several inventive and comparative denosumab formulations stored at 40° C.

### DETAILED DESCRIPTION OF THE EMBODIMENTS

Embodiments of the present disclosure will now be described more fully hereinafter with reference to the accompanying drawings, in which some, but not all of the embodiments of the disclosure are shown.

Unless defined otherwise, all technical and scientific terms used herein have the same meaning as commonly understood by one having ordinary skill in the art to which the invention pertains.

As used herein, the words “a” and “an” and the like carry the meaning of “one or more.” Additionally, within the description of this disclosure, where a numerical limit or range is stated, the endpoints are included unless stated otherwise. Also, all values and subranges within a numerical limit or range, including subranges excluding endpoints, are specifically included as if explicitly written out.

As used herein, the terms “optional” or “optionally” mean that the subsequently described event(s) can or cannot occur or the subsequently described component(s) may or may not be present (e.g., 0 wt %).

The term “comprising” is an open-ended term synonymous with terms such as including, containing, or having and is used herein to claim aspects of the invention which may include additional steps, components, functionality, and/or structure. The term “consisting essentially of” is used herein to claim aspects of the invention and additional unrecited steps or components that would not have a material effect on the basic and novel properties of a formulation or method according to the present disclosure. Basic and novel properties of the present disclosure include improvements in surfactant stability relative to known denosumab formulations. In formulations described herein, “consisting essentially of” serves to exclude the addition of unrecited actives, sugars, buffers, surfactants, etc., but would permit the addition of minor agents such as a pH adjusting agents, antioxidants, or preservatives. The term “consisting of” is used to claim aspects of the invention in which only those features explicitly recited in a claim are included and thus other steps or components not explicitly or inherently included in the claim are excluded. In formulations described herein, “consisting of” excludes all unrecited actives, sugars, buffers, surfactants, etc. For clarity, impurities of sufficiently low level so as to be pharmaceutically acceptable, e.g., those incident to pH adjustment, are not excluded.

It is expressly intended that the features of the various features of formulations and methods described herein be interchangeably used and used in combination. That is, to the extent that component, component amount, or other parameter is mentioned with respect to an embodiment, it is expressly intended that such parameter be suitably substituted for use in other embodiments. The present inventor has contemplated such combinations and intend that they be encompassed by this disclosure even if not written out.

In what follows, unless otherwise indicated, “%” will mean “% (w/v)”, calculated as [100×mx/vtot], where mx is the mass of component x in g and vtot is the total volume of all components in mL.

The term “substantially” as used herein shall mean comprising more than 50% by weight, where relevant, unless otherwise indicated.

The recitation of a numerical range using endpoints includes the endpoints and all numbers subsumed within that range (e.g., from 1 to 10 includes 1, 1.5, 2, 5, 5.75, 7.1, 8.5, 9, 10, etc.).

As used herein, the terms “about” and “approximately” are used synonymously. Both terms are meant to cover any normal fluctuations or variations understood by those skilled in the art. In some embodiments, “about” and “approximately” refers to ±10% of the reference value, or ±5%, or ±2%, or ±1%, or ±0.1% of the reference value. This disclosure is intended to encompass alternative embodiments in which numbers not modified with the terms “about” or “approximately” are so modified.

As used herein, the term “an effective amount” or “therapeutically effective amount” refers to an amount of denosumab that a will elicit a biological or medical response in a subject, for example, inhibiting a disease and its progression (for example, inhibiting a disease, condition or disorder in an individual who is experiencing or displaying the pathology or symptomatology of the disease, condition or disorder, including arresting further development of the pathology and/or symptomatology of the disease; and/or ameliorating a disease (for example, ameliorating a disease, condition or disorder in an individual who is experiencing or displaying the pathology or symptomatology of the disease, condition or disorder (for example, reducing the pathology, reversing the pathology and/or symptomatology of the disease).

As used herein, the phrase “treating”, “treat”, and “treatment” and the like includes inhibiting a disease and/or its progression (for example, inhibiting a disease, condition or disorder in an individual who is experiencing or displaying the pathology or symptomatology of the disease, condition or disorder, including arresting further development of the pathology and/or symptomatology of the disease; and/or ameliorating a disease (for example, ameliorating a disease, condition or disorder in an individual who is experiencing or displaying the pathology or symptomatology of the disease, condition or disorder (for example, reducing the pathology, reversing the pathology and/or symptomatology of the disease).

As used herein, the term “preventing”, “prevent”, and “prevention” and the like, includes the prevention of the recurrence and/or the onset of one or more symptoms of a disorder or disease, especially in a subject which has been analyzed to be susceptible or likely to develop the disease.

As used herein, the phrase “a subject in need thereof” means that the subject has been identified as having a need for the herein described treatment.

As used herein, the term “subject” and “patient” are interchangeable and may be taken to mean any living organism, which may be treated with compounds of the present invention. As such, the terms “patient” and “subject” may include a human adult or child.

All references cited herein, including patents, patent applications, publications, and databases, are hereby incorporated by reference in their entireties, whether previously specifically incorporated or not.

The inventors surprisingly discovered that certain formulations under development exhibited remarkable performance in stability of surfactant concentration—i.e., prevention of surfactant degradation—during storage relative to Prolia® and other alternative denosumab formulations. The inventors believe that surfactant stability in denosumab formulations is positively influenced by the use of a poloxamer surfactant optionally together with a histidine buffer.

Described herein is an aqueous pharmaceutical formulation, comprising 50 to 80 mg/mL denosumab, 200 to 300 mM sugar, 1 to 10 mM histidine buffer, 0.005 to 0.1% (w/v) of a poloxamer, and water. The formulation has a pH of from 5.5 to 6.5. In embodiments, the formulation includes 55 to 65 or 65 to 75 mg/mL denosumab, 230 to 250 mM sugar, 4 to 6 mM histidine buffer, 0.01 to 0.05% (w/v) poloxamer, and water, the formulation having a pH of from 6.0 to 6.4. In further embodiments, the aqueous pharmaceutical formulation includes 60 or 70 mg/mL denosumab, 240 mM sugar, 5 mM histidine buffer, 0.03% (w/v) poloxamer, and water, the formulation having a pH of 6.2.

The denosumab formulations described herein are suitable for pharmaceutical administration, such as parenteral administration, and especially for subcutaneous injection such as by a syringe, pre-filled single-use syringe, or autoinjector.

### Water

The main component (or carrier) of the denosumab formulations is water of pharmaceutical grade. “Pharmaceutical-grade water” refers to water meeting at least the purity and quality requirements suitable for making a parenteral denosumab formulation as set forth in the USP for “water for injection” (or “WFI”). As is understood by workers skilled in the art, WFI is drinking water purified by distillation, or a purification process equivalent or superior to distillation, resulting in the removal of chemicals and microorganisms. WFI contains no added substances. The pharmaceutical-grade water may be sterilized prior to and/or after being combined with other components of the denosumab formulation, but sterilization is not required.

The denosumab formulations herein comprise several components that are dissolved and/or dispersed, but typically fully dissolved, in the pharmaceutical-grade water. These include: denosumab, a sugar, a histidine buffer, and a poloxamer. In some embodiments, the denosumab formulations herein also comprise histidine buffer as the sole buffering agent exclusive of the antibody (denosumab).

### Denosumab

The formulations described herein include the antibody denosumab, whose encoded amino acid sequence contains 1326 amino acids, including any accepted variations or biosimilars thereof, including for example the denosumab sequences disclosed in WO 2018/200918, the contents of which are incorporated by reference.

In embodiments, denosumab is produced from one or more vectors encoding amino acid sequences comprising the variable light chain sequence of denosumab:

(SEQ ID NO: 1)
EIVLT QSPGT LSLSP GERAT LSCRA SQSVR GRYLA WYQQK
PKQAP RLLIY GASSR ATGIP DRFSG SGSGT DFTLT ISRLE
PEDFA VFYCQ QYGSS PRTFG QGTKV EIK

In further embodiments, denosumab is produced from one or more vectors encoding amino acid sequences comprising the light chain sequence of denosumab:

(SEQ ID NO: 2)
EIVLT QSPGT LSLSP GERAT LSCRA SQSVR GRYLA WYQQK
PKQAP RLLIY GASSR ATGIP DRFSG SGSGT DFTLT ISRLE
PEDFA VFYCQ QYGSS PRTFG QGTKV EIKRT VAAPS VTIFP
PSDEQ LKSGT ASVVC LLNNF YPREA KVQWK VDNAL QSGNS
QESVT EQDSK DSTYS LSSTL TLSKA DYEKH KVYAS EVTHQ
GLSSP VTKSF NRGEC

In further embodiments, denosumab is produced from one more vectors encoding the variable heavy chain sequence of denosumab either on the same or separate vector from the variable light chain:

(SEQ ID NO: 3)
EVQLL ESGGG LVQPG GSLRL SCAAS GFTFS SYAMS WVRQA
PGKGL EWVSG ITGSG GSTYY ADSVK GRFTI SRDNS KNTLY
LQMNS LRAED TAVYY CAKSP GTTVI MSWFD PWGQG TLVTV
SS

In further embodiments, denosumab is produced from one or more vectors encoding the heavy chain sequence of denosumab either on the same or separate vector from the light chain:

(SEQ ID NO: 4)
EVQLL ESGGG LVQPG GSLRL SCAAS GFTFS SYAMS WVRQA
PGKGL EWVSG ITGSG GSTYY ADSVK GRFTI SRDNS KNTLY
LQMNS LRAED TAVYY CAKSP GTTVI MSWFD PWGQG TLVTV
SSAST KGPSV FPLAP CSRST SESTA ALGCL VKDYF PEPVT
VSMNS GALTS GVHTF PAVLQ SSGLY SLSSV VTVPS SNFGT
QTYTC NVDHK PSNTK VDKTV ERKCC VECPP CPAPP VAGPS
VFLFP PKPKD TLMIS RTPEV TCVVV DVSHE DPEVQ FNWYV
DGVEV HNAKT KPREE QFNST FRVVS VLTVV HQDWL NGKEY
KCKVS NKGLP APIEK TISKT KGQPR EPQVY TLPPS REEMT
KNQVS LTCLV KGFYP SDIAV EWESN GQPEN NYKTT PPMLD
SDGSF FLYSK LTVDK SRWQQ GNVFS CSVMH EALHN HYTQK
SLSLS PGK

Because denosumab is produced by a biological process, certain minor variations can occur from batch to batch, including but not limited to “lysine clipping” wherein the C-terminal lysine of the constant heavy chain is clipped off, which variations are considered denosumab and have no clinically meaningful effect. A biosimilar, as is understood by workers skilled in the art, refers to a biologic that is highly similar to the reference product (in this case to the denosumab produced by Amgen Inc.) and has no clinically meaningful differences therefrom in terms of efficacy and safety. The high degree of similarity between the biosimilar and reference product generally includes the physical, chemical, and biological properties and the minor differences permitted are not clinically meaningful. This understanding is used by the United States Food and Drug Administration (U.S. FDA) and the European Medicines Agency (EMA) as well as by pharmaceutical companies seeking permission to market a biosimilar of a reference product. In the U.S., a biosimilar may be denoted with an extension after the reference product name. In the context of the disclosure herein, for the avoidance of doubt, all such extension names of denosumab should be considered within the scope of the term “denosumab” as used in herein. The concentration of denosumab in the denosumab formulations described herein may be from 50 mg/mL to 80 mg/mL, such as from 55 mg/mL to 65 mg/mL or 65 mg/mL to 75 mg/mL, and in particular, may be 60 mg/ml or may be 70 mg/mL.

### Sugar

The denosumab formulations described herein comprise sugar, preferably in amounts of 200 to 300 mM. The primary purpose of sugar is to stabilize the denosumab and modify the osmolality of the formulations to an iso-osmotic level. The sugar may be any suitable sugar. In some embodiments, the sugar is selected from sucrose, trehalose, maltose, and lactose, either individually or in any suitable combination of two or more. In one embodiment, the sugar is sucrose. Alternatively, or in addition, a sugar alcohol, such as sorbitol.

The sugar may be present in the denosumab formulations described herein at any suitable concentration from 200 to 300 mM. In some embodiments, the sugar is present at a concentration on 200 mM to 225 mM, or 225 mM to 250 mM, or 210 mM to 240 mM, or 235 mM to 245 mM, or 235 mM to 265 mM, or 250 mM to 275 mM, or 265 mM to 285 mM, or 200 mM, 205 mM, 210 mM, 215 mM, 220 mM, 225 mM, 230 mM, 235 mM, 240 mM, 245 mM, 250 mM, 255 mM, 260 mM, 265 mM, 270 mM, 275 mM, 280 mM, 285 mM, 290 mM, 295 mM, or 300 mM. In some embodiments, the sugar is present in the denosumab formulations described herein at a concentration of from 200 to 250 mM. In one embodiment, the denosumab formulation herein comprises about 240 mM sucrose.

### Poloxamer

The denosumab formulations described herein comprise a poloxamer as a surfactant. Poloxamers are nonionic triblock copolymers composed of a central hydrophobic chain of polyoxypropylene (poly(propylene oxide)) flanked by two hydrophilic chains of polyoxyethylene (poly(ethylene oxide)). Poloxamers may protect protein formulations against agitation induced stress and/or exposure of proteins to surfaces and/or air-liquid interfaces. The poloxamer used herein is not intended to be particularly limited. In a particular embodiment, the poloxamer may be poloxamer 188, otherwise known as P188, where the first two digits (18) multiplied by 100 give the approximate molecular mass of the polyoxypropylene core (i.e., 1800), and the last digit (8) multiplied by 10 gives the percentage of polyoxyethylene content (i.e., 80%).

The denosumab formulations described herein comprise a poloxamer in an amount of 0.005 to 0.1% (w/v) based on the total formulation. In some embodiments, the poloxamer is present in an amount of from 0.005% to 0.01%, or from 0.01% to 0.05%, or from 0.05% to 0.1%, or from 0.0075% to 0.0125%, or from 0.01% to 0.075%, or from 0.01% to 0.04%, or from 0.03% to 0.08%, or an amount of 0.005%, 0.0075%, 0.01%, 0.02%, 0.03%, 0.04%, 0.05%, 0.06%, 0.07%, 0.08%, 0.09% or 0.1% (w/v).

### Osmolality

The denosumab formulations described herein have an osmolality within a parenterally acceptable range, generally between 200 and 600 mOsm/kg. Typically, the osmolality of the denosumab formulations described herein is within the range of between 240 and 400 mOsm/kg. In some embodiments, the denosumab formulations have an osmolality within the range of 260 to 380 mOsm/kg, and typically between 270 and 360 mOsm/kg. In other embodiments, the osmolality is within the range of 285 to 345 mOsm/kg. Typically, values within these ranges can be attained by balancing the kind and amount of the sugar, the amount of the histidine buffer, and to a lesser extent, the kind and amount of poloxamer, given the amount of denosumab in pharmaceutical-grade water. Osmolality may also be adjusted by adding additional agents such as salts (e.g., NaCl).

### pH

The denosumab formulations described herein have a pH of 5.5 to 6.5. In embodiments, the pH is 5.6 to 5.8, 6.1 to 6.3, about 5.7 to about 6.2, or any subrange therein. In further embodiments, the pH is 5.7, about 5.7, 6.2, or about 6.2. The pH can be adjusted, if necessary, by the addition of pH adjustment agents. In some embodiments, the pH adjustment agents may be an acid or a base. In some embodiments, the acid is HCl or acetic acid. In some embodiments, the base is NaOH.

### Histidine Buffer

The denosumab formulations herein may comprise a buffer, preferably a histidine buffer, and preferably at a concentration of 1 to 10 mM. The concentration of histidine buffer does not necessarily refer to the concentration of starting ingredients used to make the buffered solution. Instead, it refers to the concentration of histidine species in the buffered solution, after the starting ingredients have been added and have undergone ionic dissociation in the equilibrated buffered solution. For example, in a denosumab formulation may include as starting ingredients L-histidine and L-histidine monohydrochloride monohydrate. Upon dissociation in the buffered solution, these concentrations of ingredients result in a histidine buffer concentration of 1 to 10 mM.

In some embodiments, the denosumab formulations herein comprise 1 to 10 mM histidine buffer. In other embodiments, the formulations comprise 3 to 7 mM histidine buffer. In other embodiments, the formulations comprise 4 to 6 mM histidine buffer. In some embodiments, the formulations comprise 4 mM, 5 mM, about 5 mM, or 6 mM histidine buffer. It is understood that an antibody such as denosumab may contribute to the buffering capacity of a formulation.

### Stability

The denosumab formulations described herein are relatively stable. Typically, the formulation has satisfactory bulk stability for at least 36 months, or at least 24 months, or at least 12 months, or at least 6 months. In general, such stability includes chemical and physical stability of denosumab such that only minor amounts of both fragmentation and aggregation of the antibody are observed.

The denosumab formulations described herein exhibit remarkable stability in surfactant concentration or, put another way, resistance to surfactant degradation. In embodiments, a denosumab formulation exhibits minimal loss of surfactant concentration during storage. Loss of surfactant concentration can be expressed as a percentage of original surfactant concentration, determined by comparing the surfactant concentration after storage under specified conditions to the concentration of the denosumab formulation as prepared. Surfactant concentration can be measured after storage, e.g., by ultra-performance liquid chromatography coupled with evaporative light scattering detection (UPLC/ELSD).

As a non-limiting, exemplary explanation, surfactant stability of the denosumab formations described herein may result, in part, from increased resistance to oxidation and hydrolysis, both enzymatically from residual host cell proteins (e.g., lipases) which are not removed during clarification and purification and/or from histidine (e.g., in combination with trace metals).

In embodiments, the denosumab formulations exhibit a loss of surfactant concentration after storage at 5° C. for 1 month of 15% or less. In embodiments, the denosumab formulations exhibit a loss of surfactant concentration after storage at 25° C. for 1 month of 40% or less, 35% or less, 25% or less, 20% or less, 15% or less, or 10% or less. In embodiments, the denosumab formulations exhibit a loss of surfactant concentration after storage at 40° C. for 1 month of 50% or less, 55% or less, 40% or less, 35% or less, 30% or less, 25% or less, 20% or less, or 15% or less. Loss of surfactant concentration may be measured, e.g., as described in the examples below.

### Formulations

In an embodiment, the denosumab formulation comprises: 50 to 80 mg/ml denosumab; 200 to 300 mM sugar; 1 to 10 mM histidine buffer; 0.005 to 0.1% (w/v) poloxamer; and water, and has a pH of from 5.5 to 6.5.

In an embodiment, the denosumab formulation comprises: about 60 mg/ml denosumab; about 240 mM sucrose; about 5 mM histidine buffer; about 0.03% (w/V) poloxamer 188; and water; and has a pH of about 6.2.

In an embodiment, the denosumab formulation consists essentially of: about 60 mg/mL denosumab; about 240 mM sucrose; about 5 mM histidine buffer; about 0.03% (w/v) poloxamer 188; and water; and has a pH of about 6.2.

In an embodiment, the denosumab formulation consists of: about 60 mg/mL denosumab; about 240 mM sucrose; about 5 mM histidine buffer; about 0.03% (w/v) poloxamer 188; and water; and has a pH of about 6.2.

In an embodiment, the denosumab formulation comprises: 60 mg/mL denosumab; 240 mM sucrose; 5 mM histidine buffer; 0.03% (w/v) poloxamer 188; and water; and has a pH of 6.2.

In an embodiment, the denosumab formulation consists essentially of: 60 mg/mL denosumab; 240 mM sucrose; 5 mM histidine buffer; 0.03% (w/v) poloxamer 188; and water; and has a pH of 6.2.

In an embodiment, the denosumab formulation consists of: 60 mg/ml denosumab; 240 mM sucrose; 5 mM histidine buffer; 0.03% (w/v) poloxamer 188; and water; and has a pH of 6.2.

In an embodiment, the denosumab formulation comprises: about 70 mg/ml denosumab; about 240 mM sucrose; about 5 mM histidine buffer; about 0.03% (w/v) poloxamer 188; and water; and has a pH of about 6.2.

In an embodiment, the denosumab formulation consists essentially of: about 70 mg/mL denosumab; about 240 mM of sucrose; about 5 mM histidine buffer; about 0.03% (w/v) poloxamer 188; and water; and has a pH of about 6.2.

In an embodiment, the denosumab formulation consists of: about 70 mg/ml denosumab; about 240 mM sucrose; about 5 mM histidine buffer; about 0.03% (w/v) poloxamer 188; and water; and has a pH of about 6.2.

In an embodiment, the denosumab formulation comprises: 70 mg/ml denosumab; 240 mM sucrose; 5 mM histidine buffer; 0.03% (w/v) poloxamer 188; and water; and has a pH of 6.2.

In an embodiment, the denosumab formulation consists essentially of: 70 mg/mL denosumab; 240 mM sucrose; 5 mM histidine buffer; 0.03% (w/v) poloxamer 188; and water; and has a pH of 6.2.

In an embodiment, the denosumab formulation consists of: 70 mg/mL denosumab; 240 mM sucrose; 5 mM histidine buffer; 0.03% (w/v) poloxamer 188; and water; and has a pH of 6.2.

In addition to the specific formulation variants identified above, the present disclosure is more broadly directed to denosumab formulations that achieve the desirable effects described herein. For example, denosumab formulations including a poloxamer surfactant optionally together with a histidine buffer may provide improved surfactant stability.

In embodiments, an aqueous pharmaceutical formulation comprises denosumab and a poloxamer. Optionally, the formulation may comprise 50 to 80 mg/mL denosumab. Optionally, the poloxamer may be poloxamer 188. Optionally, the formulation may comprise comprising 0.005 to 0.1% (w/v) poloxamer. Optionally the formulation may comprise a histidine buffer. Optionally, the formulation may comprise 1 to 10 mM histidine buffer. Optionally, the formulation may comprise a sugar. Optionally, the formulation may comprise sucrose. Optionally, the formulation may comprise 200 to 300 mM sugar. Optionally the formulation may have a pH of 5.5 to 6.5. In addition to these options, the formulation may include any combination of the components, component amounts, and properties described herein including, particularly, aspects of the specific formulation variants enumerated above.

In embodiments, a denosumab formulation for parenteral injection comprises a sugar or sugar alcohol, a buffer, and a surfactant, wherein the surfactant is a poloxamer. Optionally, the formulation may comprise 50 to 80 mg/ml denosumab. Optionally, the poloxamer may be poloxamer 188. Optionally, the formulation may comprise comprising 0.005 to 0.1% (w/v) poloxamer. Optionally the formulation may comprise a histidine buffer. Optionally, the formulation may comprise 1 to 10 mM histidine buffer. Optionally, the formulation may comprise a sugar. Optionally, the formulation may comprise sucrose. Optionally, the formulation may comprise 200 to 300 mM sugar. Optionally the formulation may have a pH of 5.5 to 6.5. In addition to these options, the formulation may include any combination of the components, component amounts, and properties described herein including, particularly, aspects of the specific formulation variants enumerated above.

In embodiments, an aqueous pharmaceutical formulation comprises denosumab and a surfactant, wherein the formulation exhibits a loss of surfactant concentration of 40% or less, when stored for one month at 25° C. Optionally, the formulation exhibits a loss of surfactant concentration of 15% or less. Optionally, the formulation comprises a poloxamer. Optionally, the formulation comprises a histidine buffer. In addition to these options, the formulation may include any combination of the components, component amounts, and properties described herein including, particularly, aspects of the specific formulation variants enumerated above.

In embodiments, an aqueous pharmaceutical formulation comprises denosumab and a surfactant, wherein the formulation exhibits a loss of surfactant concentration of 50% or less, when stored for one month at 40° C. Optionally, the formulation exhibits a loss of surfactant concentration of 15% or less. Optionally, the formulation comprises a poloxamer. Optionally, the formulation comprises a histidine buffer. In addition to these options, the formulation may include any combination of the components, component amounts, and properties described herein including, particularly, aspects of the specific formulation variants enumerated above.

### Manufacture of Formulation

The denosumab formulations described herein can be made by combining denosumab, a sugar, a histidine buffer, and a poloxamer, in a pharmaceutical-grade water to form a solution and/or suspension. The order of addition and method of combining is normally not of particular importance. For convenience and commercial practicality, denosumab may be formulated into the formulations described herein using well known ultrafiltration/diafiltration (UF/DF) techniques. In general, the expressed denosumab is captured and purified by various processes and then subjected to UF/DF for additional purification, concentration, and modification of the liquid media. The denosumab solution resulting from the previous purification process steps can be replaced using diafiltration, as is well known in the art. In making the formulations herein described, denosumab can be diafiltered with a solution of a sugar and histidine buffer, in WFI, with the poloxamer being added during or after the completion of the UF/DF process. The poloxamer may be added as a solution in WFI or in the diafiltering solution. The pH can be adjusted, if needed, before, during, or after UF/DF operations such as by the introduction of HCl or other pH adjusting agent that is suitable for a parenteral formulation. The denosumab formulation is not, however, limited to the use of UF/DF and any suitable process or means for combining the recited ingredients in the desired concentrations can be used to make the denosumab formulations described herein.

### Uses of Formulation

The denosumab formulations described herein are useful for preventing or treating a variety of diseases and disorders or symptoms thereof that respond to denosumab or anti-RANKL antibody treatment. These include osteoporosis, loss of bone mass, skeletal issues in multiple myeloma patients or patients with solid tumor bone metastases, giant cell tumors of the bone, and hypercalcemia; particularly, treatment of postmenopausal women with osteoporosis at high risk for fracture, treatment to increase bone mass in men at high risk for fracture receiving androgen deprivation therapy for nonmetastatic prostate cancer, treatment to increase bone mass in women at high risk for fracture receiving adjuvant aromatase inhibitor therapy for breast cancer, prevention of skeletal-related events in patients with bone metastases from solid tumors, treatment of adults and skeletally mature adolescents with giant cell tumor of bone that is unresectable or where surgical resection is likely to result in severe morbidity.

In embodiments, the present disclosure relates to methods of treating or preventing the foregoing diseases or conditions, in which an effective amount of a formulation as described herein is administered to subject, for example a human patient, in need of such treatment or prevention. In embodiments, such administration may subcutaneous injection of a predetermined amount of a formulation as described herein. Such subcutaneous injection may be carried out by the subject (patient) his/her/their self or by a caregiver, using a syringe filled from a vial, a prefilled syringe, or an autoinjector. Subcutaneous injection may be in the upper arm, upper thigh, or abdomen of the subject. The vial, prefilled syringe, or autoinjector may contain, for example 1.0 or 1.7 mL of a formulation as described herein. Administration of a formulation as described herein may include co-administration with calcium (e.g., 1,000 mg daily) and/or vitamin D (e.g., 400 IU daily).

The dosing amount and regimen are well known from the administration of Prolia® and Xgeva® as, for example, approved by the U.S. FDA or EMA. As noted above, for example, the denosumab formulations herein may be loaded into a prefilled syringe to provide 1.0 mL of the formulation, corresponding to a denosumab dose of 60 mg, or may be provided in a vial for injection in an amount of 1.7 mL of the formulation, corresponding to a denosumab dose of 120 mg. Exemplary dosage regimens include: administering 120 mg every 4 weeks as a subcutaneous injection in the upper arm, upper thigh, or abdomen (e.g., for treating bone metastasis from solid tumors); administering 120 mg every 4 weeks as a subcutaneous injection in the upper arm, upper thigh, or abdomen with additional 120 mg doses on days 8 and 15 of the first month of therapy (e.g., for treating giant cell tumor of bone); and administering 60 mg every 6 months as a subcutaneous injection in the upper arm, upper thigh, or abdomen.

In various exemplary embodiments, the present disclosure relates the following items and all combinations thereof:
1. An aqueous pharmaceutical formulation, comprising:
50 to 80 mg/mL denosumab;
200 to 300 mM sugar;
1 to 10 mM histidine buffer;
0.005 to 0.1% (w/v) poloxamer; and
water;
wherein the formulation has a pH of from 5.5 to 6.5.
2. The formulation of item 1 or any item depending therefrom, comprising:
about 60 mg/mL denosumab;
about 240 mM sucrose;
about 5 mM histidine buffer;
about 0.03% (w/v) poloxamer 188; and
water;
wherein the formulation has a pH of about 6.2.
3. The formulation of item 2 or any item depending therefrom, consisting essentially of:
about 60 mg/mL denosumab;
about 240 mM sucrose;
about 5 mM histidine buffer;
about 0.03% (w/v) poloxamer 188; and water.
4. The formulation of item 2 or any item depending therefrom, consisting of:
about 60 mg/mL denosumab;
about 240 mM sucrose;
about 5 mM histidine buffer;
about 0.03% (w/v) poloxamer 188; and
water.
5. The formulation of item 1 or any item depending therefrom, comprising:
60 mg/mL denosumab;
240 mM sucrose;
5 mM histidine buffer;
0.03% (w/v) poloxamer 188; and
water;
wherein the formulation has a pH of 6.2.
6. The formulation of item 5 or any item depending therefrom, consisting essentially of:
60 mg/mL denosumab;
240 mM sucrose;
5 mM histidine buffer;
0.03% (w/v) poloxamer 188; and
water.
7. The formulation of item 5 or any item depending therefrom, consisting of:
60 mg/mL denosumab;
240 mM sucrose;
5 mM histidine buffer;
0.03% (w/v) poloxamer 188; and
water.
8. The formulation of item 1 or any item depending therefrom, comprising:
about 70 mg/mL denosumab;
about 240 mM sucrose;
about 5 mM histidine buffer;
about 0.03% (w/v) poloxamer 188; and
water;
wherein the formulation has a pH of about 6.2.
9. The formulation of item 8 or any item depending therefrom, consisting essentially of:
about 70 mg/mL denosumab;
about 240 mM of sucrose;
about 5 mM histidine buffer;
about 0.03% (w/v) poloxamer 188; and
water.
10. The formulation of item 8 or any item depending therefrom, consisting of:
about 70 mg/mL denosumab;
about 240 mM sucrose;
about 5 mM histidine buffer;
about 0.03% (w/v) poloxamer 188; and
water.
11. The formulation of item 1 or any item depending therefrom, comprising:
70 mg/mL denosumab;
240 mM sucrose;
5 mM histidine buffer;
0.03% (w/v) poloxamer 188; and
water;
wherein the formulation has a pH of 6.2.
12. The formulation of item 11 or any item depending therefrom, consisting essentially of:
70 mg/mL denosumab;
240 mM sucrose;
5 mM histidine buffer;
0.03% (w/v) poloxamer 188; and
water.
13. The formulation of item 11 or any item depending therefrom, consisting of:
70 mg/mL denosumab;
240 mM sucrose;
5 mM histidine buffer;
0.03% (w/v) poloxamer 188; and
water.
14. The formulation of item 1 or any item depending therefrom, comprising 60 to 70 mg/mL denosumab.
15. The formulation of item 1 or any item depending therefrom, comprising from 220 to 260 mM sugar.
16. The formulation of item 1 or any item depending therefrom, comprising from 0.1 to 0.5% (w/v) poloxamer.
17. The formulation of item 1 or any item depending therefrom, wherein the sugar is at least one selected from the group consisting of sucrose, trehalose, maltose, and lactose.
18. The formulation of item 1 or any item depending therefrom, wherein the sugar is sucrose.
19. The formulation of item 1 or any item depending therefrom, wherein the poloxamer is poloxamer 188.
20. The formulation of item 1 or any item depending therefrom, wherein the formulation has a pH of about 5.7 to about 6.2.
21. The formulation of item 1 or any item depending therefrom, wherein the formulation has a pH of about 5.7.
22. The formulation of item 1 or any item depending therefrom, wherein the formulation has a pH of about 6.2.
23. An aqueous pharmaceutical formulation, comprising denosumab and a poloxamer.
24. The formulation of item 23 or any item depending therefrom, comprising 50 to 80 mg/mL denosumab.
25. The formulation of item 23 or any item depending therefrom, wherein the poloxamer is poloxamer 188.
26. The formulation of item 23 or any item depending therefrom, comprising 0.005 to 0.1% (w/v) poloxamer.
27. The formulation of item 23 or any item depending therefrom, further comprising a histidine buffer.
28. The formulation of item 27 or any item depending therefrom, comprising 1 to 10 mM histidine buffer.
29. The formulation of item 23 or any item depending therefrom, further comprising a sugar.
30. The formulation of item 29 or any item depending therefrom, wherein the sugar is sucrose.
31. The formulation of item 29 or any item depending therefrom, comprising 200 to 300 mM sugar.
32. The formulation of item 23 or any item depending therefrom, having a pH of 5.5 to 6.5.
33. A denosumab formulation for parenteral injection, comprising a sugar or sugar alcohol, a buffer, and a surfactant, wherein the surfactant is a poloxamer.
34. The formulation of item 33 or any item depending therefrom, comprising 50 to 80 mg/mL denosumab.
35. The formulation of item 33 or any item depending therefrom, wherein the poloxamer is poloxamer 188.
36. The formulation of item 33 or any item depending therefrom, comprising 0.005 to 0.1% (w/v) poloxamer.
37. The formulation of item 33 or any item depending therefrom, wherein the buffer is a histidine buffer.
38. The formulation of item 37 or any item depending therefrom, comprising 1 to 10 mM histidine buffer.
39. The formulation of item 33 or any item depending therefrom, wherein the sugar or sugar alcohol is sucrose.
40. The formulation of item 33 or any item depending therefrom, comprising 200 to 300 mM sugar or sugar alcohol.
41. The formulation of item 33 or any item depending therefrom, having a pH of 5.5 to 6.5.
42. An aqueous pharmaceutical formulation, comprising denosumab and a surfactant, wherein the formulation exhibits a loss of surfactant concentration of 40% or less, when stored for one month at 25° C.
43. The formulation of item 42 or any item depending therefrom, wherein the loss of surfactant concentration is 15% or less.
44. The formulation of item 42 or any item depending therefrom, wherein the surfactant comprises a poloxamer.
45. The formulation of item 44 or any item depending therefrom, further comprising a histidine buffer.
46. The formulation of item 45 or any item depending therefrom, comprising:
50 to 80 mg/mL denosumab;
200 to 300 mM sugar;
1 to 10 mM histidine buffer;
0.005 to 0.1% (w/v) poloxamer; and
water;
wherein the formulation has a pH of from 5.5 to 6.5.
47. The formulation of item 46 or any item depending therefrom, consisting essentially of:
60 mg/mL denosumab;
240 mM sucrose;
5 mM histidine buffer;
0.03% (w/v) poloxamer 188; and
water;
wherein the formulation has a pH of 5.7 to 6.2.
48. An aqueous pharmaceutical formulation, comprising denosumab and a surfactant, wherein the formulation exhibits a loss of surfactant concentration of 50% or less, when stored for one month at 40° C.
49. The formulation of item 48 or any item depending therefrom, wherein the loss of surfactant concentration is 15% or less.
50. The formulation of item 48 or any item depending therefrom, wherein the surfactant comprises a poloxamer.
51. The formulation of item 50 or any item depending therefrom, further comprising a histidine buffer.
52. The formulation of item 51 or any item depending therefrom, comprising:
50 to 80 mg/mL denosumab;
200 to 300 mM sugar;
1 to 10 mM histidine buffer;
0.005 to 0.1% (w/v) poloxamer; and
water;
wherein the formulation has a pH of from 5.5 to 6.5.
53. The formulation of item 52 or any item depending therefrom, consisting essentially of:
60 mg/mL denosumab;
240 mM sucrose;
5 mM histidine buffer;
0.03% (w/v) poloxamer 188; and
water;
wherein the formulation has a pH of 5.7 to 6.2.
54. The formulation of any one of items 1 to 53 or any item depending therefrom for use in treating or preventing osteoporosis or loss of bone mass.
55. The formulation of any one of items 1 to 53 or any item depending therefrom for use in treating or preventing skeletal-related events associated with multiple myeloma, solid tumor bone metastases, or giant cell tumors of the bone.
56. The formulation of any one of items 1 to 53 or any item depending therefrom for use in treating or preventing hypercalcemia.
57. A method for treating osteoporosis or loss of bone mass, comprising administering the formulation of any one of items 1 to 53 or any item depending therefrom to a subject in need of such treatment.
58. A method for treating a skeletal-related event associated with multiple myeloma, solid tumor bone metastases, or giant cell tumors of the bone, comprising administering the formulation of any one of items 1 to 53 or any item depending therefrom to a subject in need of such treatment.
59. A method for treating hypercalcemia, comprising administering the formulation of any one of items 1 to 53 or any item depending therefrom to a subject in need of such treatment.
60. A method for preventing osteoporosis or loss of bone mass, comprising administering the formulation of any one of items 1 to 53 or any item depending therefrom to a subject in need of such treatment.
61. A method for preventing a skeletal-related event associated with multiple myeloma, solid tumor bone metastases, or giant cell tumors of the bone, comprising administering the formulation of any one of items 1 to 53 or any item depending therefrom to a subject in need of such treatment.
62. A method for preventing hypercalcemia, comprising administering the formulation of any one of items 1 to 53 or any item depending therefrom to a subject in need of such treatment.
63. Use of the formulation of any one of items 1 to 53 or any item depending therefrom in the manufacture of a medicament for treating or preventing osteoporosis or loss of bone mass.
64. Use of the formulation of any one of items 1 to 53 or any item depending therefrom in the manufacture of a medicament for treating or preventing skeletal-related events associated with multiple myeloma, solid tumor bone metastases, or giant cell tumors of the bone.
65. Use of the formulation of any one of items 1 to 53 or any item depending therefrom in the manufacture of a medicament for treating or preventing hypercalcemia.

### EXAMPLES

The present invention will now be described with reference to the following examples which should be considered in all respects as illustrative and non-restrictive. It will be appreciated by those skilled in the art that embodiments of the present disclosure may be embodied in many other forms; in particular, features of any one of the various described examples may be provided in any combination in any of the other described examples.

Aqueous denosumab formulations, Examples 1 and 2 (E1 and E2) and Comparative Examples 1 to 6 (CE1 to CE6), were prepared having compositions as specified in TABLE 1A and TABLE 1B below. E1 and E2 are inventive denosumab formulations including a poloxamer surfactant. CE1 approximates Prolia®. CE2 to CE6 are alternative denosumab formulations including a polysorbate surfactant. CE5 and CE6 differ from CE2 and CE3 in that a post-harvest filtration step was omitted in preparing the denosumab used in CE5 and CE6.

TABLE 1A

| Example | | E1 | E2 | CE1 | CE2 |
|---|---|---|---|---|---|
| Denosumab (mg/mL) | | 60 | 60 | 60 | 60 |
| Buffer | Histidine | 5 | 5 | — | 5 |
| System (mM) | Acetate | — | — | 17 | — |
| Sugar (mM) | Sucrose | 240 | 240 | — | 240 |
| | Sorbitol | — | — | 258 | — |
| Surfactant | P188 | 0.03 | 0.03 | — | — |
| (% (w/v)) | PS20 | — | — | 0.01 | — |
| | PS80 | — | — | — | 0.02 |

| | | | | |
|---|---|---|---|---|
| EDTA (% (w/v)) | — | — | — | — |
| pH | 5.7 | 6.2 | 5.2 | 5.7 |

TABLE 1B

| Example | | CE3 | CE4 | CE5 | CE6 |
|---|---|---|---|---|---|
| Denosumab (mg/mL) | | 60 | 60 | 60 | 60 |
| Buffer | Histidine | 5 | 5 | 5 | 5 |
| System (mM) | Acetate | — | — | — | — |
| Sugar (mM) | Sucrose | 240 | 240 | 240 | 240 |
| | Sorbitol | — | — | — | — |
| Surfactant | P188 | — | — | — | — |
| (% (w/v)) | PS20 | — | — | — | — |
| | PS80 | 0.02 | 0.03 | 0.02 | 0.02 |

| | | | | |
|---|---|---|---|---|
| EDTA (% (w/v)) | — | 0.005 | — | — |
| pH | 6.2 | 6.2 | 5.7 | 6.2 |

Surfactant concentration was monitored for denosumab formulations E1, E2, and CE1 to CE6 at various storage temperatures over various periods of time. For each formulation, vials were prepared with a fill volume of approximately 0.5 mL, stored at the specified conditions, analyzed at the specified pull points, and returned to the specified storage conditions after the analysis. Surfactant concentration was detected at the indicated times by ultra-performance liquid chromatography coupled with evaporative light scattering detection (UPLC/ELSD).

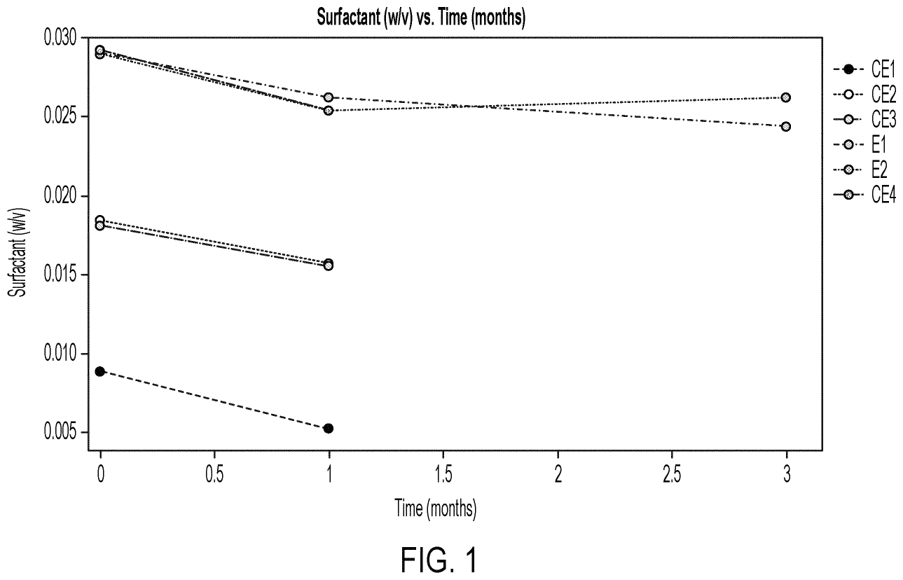

Denosumab formulations E1, E2, and CE1 to CE4 were stored at 5° C. and the surfactant concentration was measured for all of the formulations after 1 month and for E1 and E2 after 3 months. FIG. 1 is a graph showing the measured surfactant concentrations, and TABLE 2A shows the calculated percent surfactant remaining at the time of each measurement.

TABLE 2A

Storage Time

| Example | Surfactant | 1 Month | 3 Months |
|---|---|---|---|
| E1 | P188 | 90% | 84% |
| E2 | P188 | 88% | 90% |
| CE1 | PS20 | 59% | — |
| CE2 | PS80 | 85% | — |
| CE3 | PS80 | 86% | — |
| CE4 | PS80 | 87% | — |

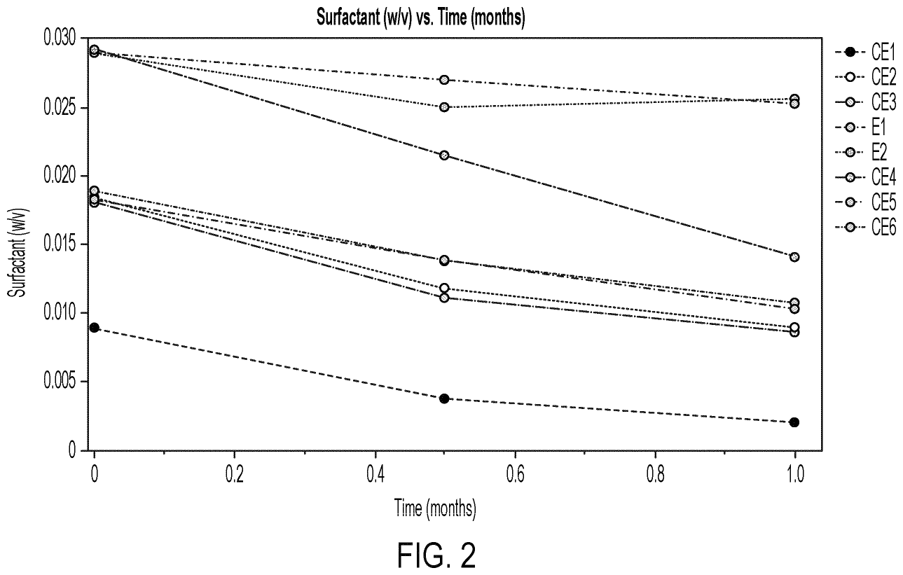

Denosumab formulations E1, E2, and CE1 to CE6 were stored at 25° C. and the surfactant concentration was measured for all of the formulations after 0.5 months and 1 month. FIG. 2 is a graph showing the measured surfactant concentrations, and TABLE 2B shows the calculated percent surfactant remaining at the time of each measurement.

TABLE 2B

Storage Time

| Example | Surfactant | 0.5 Months | 1 Month |
|---|---|---|---|
| E1 | P188 | 93% | 87% |
| E2 | P188 | 86% | 88% |
| CE1 | PS20 | 42% | 23% |
| CE2 | PS80 | 64% | 48% |
| CE3 | PS80 | 61% | 48% |
| CE4 | PS80 | 74% | 48% |
| CE5 | PS80 | 75% | 56% |
| CE6 | PS80 | 73% | 57% |

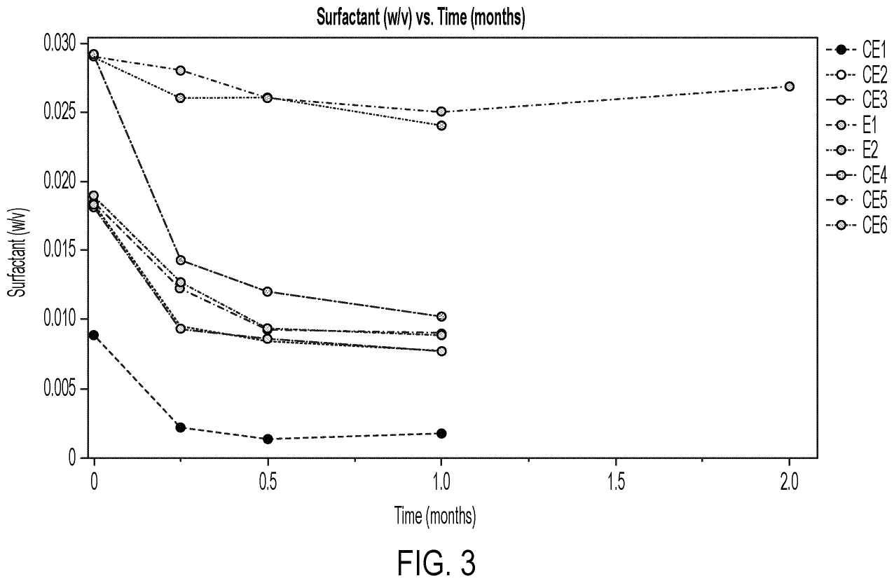

Denosumab formulations E1, E2, and CE1 to CE4 were stored at 40° C. and the surfactant concentration was measured for all of the formulations after 0.25 months, 0.5 months, and 1 month and for E1 after 2 months. FIG. 3 is a graph showing the measured surfactant concentrations, and TABLE 2C shows the calculated percent surfactant remaining at the time of each measurement.

TABLE 2C

| | Surfac- | Storage Time | | | |
|---|---|---|---|---|---|
| Example | tant | 0.25 Months | 0.5 Months | 1 Month | 2 Months |
| E1 | P188 | 97% | 90% | 86% | 92% |
| E2 | P188 | 90% | 90% | 83% | — |
| CE1 | PS20 | 24% | 15% | 19% | — |
| CE2 | PS80 | 51% | 46% | 41% | — |
| CE3 | PS80 | 51% | 47% | 47% | — |
| CE4 | PS80 | 49% | 41% | 35% | — |
| CE5 | PS80 | 67% | 50% | 49% | — |
| CE6 | PS80 | 67% | 49% | 47% | — |

As is apparent from the results above, denosumab formulations E1 and E2, which employed poloxamer 188 as a surfactant, exhibited remarkable performance in stability of surfactant concentration—i.e., prevention of surfactant degradation—during storage relative to the Prolia® formulation, which included polysorbate 20 as a surfactant, and the alternative denosumab formulations including polysorbate 80 as a surfactant. Excellent performance was seen at intended (5° C.), accelerated (25° C.), and stressed (40° C.) storage conditions.

In some instances, surfactant concentration exhibited minor increases over time, which suggests some variability in the evaluations. Comparison of CE2 and CE3 with CE5 and CE6 indicates that the omitted filtration step in CE5 and CE6 did not have a sustained effect on surfactant concentration. The results for CE4 indicate that addition of EDTA did not have a beneficial impact on surfactant concentration.

The foregoing discussion discloses and describes merely exemplary embodiments of the present disclosure. As will be understood by those skilled in the art, the present disclosure may be embodied in other specific forms without departing from the spirit or essential characteristics thereof. Accordingly, the disclosure of the present invention is intended to be illustrative, but not limiting of the scope of the disclosure, as well as other claims. The disclosure, including any readily discernible variants of the teachings herein, defines, in part, the scope of the foregoing claim terminology such that no inventive subject matter is dedicated to the public.

1. An aqueous pharmaceutical formulation, comprising denosumab and a poloxamer.

2. The aqueous formulation as claimed in claim 1, comprising about 50 to about 80 mg/mL denosumab.

3. The aqueous formulation as claimed in claim 1, wherein the poloxamer is poloxamer 188.

4. The aqueous formulation as claimed in claim 1, comprising about 0.005 to about 0.1% (w/v) poloxamer.

5. The aqueous formulation as claimed in claim 1, further comprising a histidine buffer.

6. The aqueous formulation as claimed in claim 1, further comprising a sugar or sugar alcohol in an amount of about 200 to about 300 mM.

7. The aqueous formulation as claimed in claim 1, having a pH of about 5.5 to about 6.5.

8. The aqueous formulation as claimed in claim 1, comprising:
about 60 or about 70 mg/mL denosumab;
about 240 mM sucrose;
about 5 mM histidine buffer;
about 0.03% (w/v) poloxamer 188;
water; and
wherein the formulation has a pH of about 6.2.

9. The aqueous formulation as claimed in claim 1, formulated for parenteral injection.

10. The aqueous formulation as claimed claim 1, wherein the formulation exhibits a loss of surfactant concentration of 40% or less, when stored for one month at 25° C.

11. The aqueous formulation as claimed in claim 1, wherein the formulation exhibits a loss of surfactant concentration of 50% or less, when stored for one month at 40° C.

12. A method for treating at least one selected from the group consisting of osteoporosis, loss of bone mass, skeletal-related events associated with multiple myeloma, solid tumor bone metastases, giant cell tumors of the bone, and hypercalcemia comprising administering the aqueous formulation as claimed in of claim 1 to a subject in need of such treatment.
